(19) United States
(12) Patent Application Publication
(10) Pub. No.: US 2026/0165709 A1
(43) Pub. Date: Jun. 18, 2026

(54) ELECTROLYTIC VASO-OCCLUSIVE DEVICE WITH IMPROVED DELIVERABILITY AND ENHANCED MRI FOLLOW-UP

(71) Applicants: STRYKER CORPORATION, Portage, MI (US); STRYKER EUROPEAN OPERATIONS LIMITED, Carrigtwohill (IE)

(72) Inventors: Hancun Chen, San Ramon, CA (US); Andrew S. Lee, San Jose, CA (US); Jimmy Dao, Portage, MI (US)

(73) Assignees: STRYKER CORPORATION, Portage, MI (US); STRYKER EUROPEAN OPERATIONS LIMITED, Carrigtwohill (IE)

(21) Appl. No.: 19/534,707

(22) Filed: Feb. 9, 2026

Related U.S. Application Data
(63) Continuation of application No. PCT/US2024/043298, filed on Aug. 21, 2024.
(60) Provisional application No. 63/578,975, filed on Aug. 25, 2023.

(51) Int. Cl. A61B 17/12

(52) U.S. Cl. CPC A61B 17/12109; A61B 2017/1205

(57) ABSTRACT

A vaso-occlusive assembly comprises a vaso-occlusive device configured for implantation in an aneurysmal sac, the vaso-occlusive device having a delivery configuration when restrained within a delivery catheter and having a deployed configuration when released from the delivery catheter into the aneurysmal sac. The vaso-occlusive assembly further comprises a delivery wire having a distal end to which the vaso-occlusive device is affixed. The delivery wire has an electrolytically severable joint proximal to the vaso-occlusive device. At least a portion of the electrolytically severable joint is composed of molybdenum (Mo) or an alloy thereof.

### RELATED APPLICATION DATA

The application is a continuation of International Patent Application No. PCT/US2024/043298, filed on Aug. 21, 2024, which claims the benefit of U.S. Provisional Patent Application Ser. No. 63/578,975, filed on Aug. 25, 2023, the entire disclosures of all of which are hereby incorporated herein by reference in their entirety into the present application.

### FIELD OF THE INVENTION

The present disclosure relates generally to medical devices and intravascular medical procedures, and more particularly, to devices and methods for occluding vascular defects, such as aneurysms.

### BACKGROUND

Vaso-occlusive devices or implants are used for a wide variety of reasons, including treatment of intravascular aneurysms. An intravascular aneurysm is a localized, blood-filled, dilation of a blood vessel that typically assumes a sac or balloon-like configuration that extends from a blood vessel and is caused by disease, blood flow/pressure exerted in the vessel, and/or weakening of the vessel wall. Intravascular aneurysms may pose a risk to a patient's health due to rupture, clotting, or dissection, which may cause hemorrhage, stroke (e.g., an intracranial aneurysm) and other damaging consequences to the patient. Approximately 25,000 intracranial aneurysms rupture each year in North America.

Commonly used vaso-occlusive devices include soft, helically wound coils formed by winding a platinum (or platinum alloy) wire strand about a “primary” mandrel. The coil is then wrapped around a larger, “secondary” mandrel, and heat treated to impart a secondary shape. For example, U.S. Pat. No. 4,994,069, issued to Ritchart et al., which is fully incorporated herein by reference as though set forth in full, describes a vaso-occlusive device that assumes a linear, helical primary shape when stretched for placement through the lumen of a delivery catheter, and a folded, convoluted secondary shape when released from the delivery catheter and deposited in the vasculature. In order to better frame and fill aneurysms, complex three-dimensional secondary shapes can be imparted on vaso-occlusive devices and the stiffness/flexibility of vaso-occlusive devices can be modified.

There are a variety of approaches to treat a ruptured or non-ruptured aneurysm including, e.g., an endovascular approach that involves delivering vaso-occlusive devices through an endovascular catheter into the aneurysm. Vaso-occlusive devices are commonly composed of self-expanding materials, so that when the devices are deployed from the delivery system into the target location in a patient, the unconstrained devices expand without requiring assistance. Self-expanding vaso-occlusive devices may be biased so as to expand upon release from the delivery catheter and/or include a shape-memory component that allows the device to expand upon exposure to a predetermined condition. Some vaso-occlusive devices may be characterized as hybrid devices, which have some characteristics of both self-expandable materials and non-self-expandable materials.

A typical endovascular approach for delivering a vaso-occlusive device into an aneurysm includes two major steps.

The first step involves positioning a small profile delivery catheter or micro-catheter at an aneurysm site using a guidewire. Typically, the distal end of the delivery catheter is provided, either by the attending physician or by the manufacturer, with a selected pre-shaped bend, e.g., 45°, 26°, “J”, “S”, or other bending shape, depending on the particular anatomy of the patient, so that it will stay in a desired position for releasing one or more vaso-occlusive device(s) into the aneurysmal sac once the guidewire is withdrawn. It is desirable that the lumen, and thus the outer diameter, of the delivery catheter be as small as possible to allow the aneurysm to be accessed through a very small vasculature.

The second step involves loading a vaso-occlusive device into the delivery catheter via a delivery device (e.g., a delivery wire) in a collapsed or radially compressed delivery configuration and then introduced into an aneurysmal sac. In some embodiments, multiple vaso-occlusive devices (e.g., two) may be concurrently loaded and then serially introduced into the aneurysmal sac. Once delivered within the aneurysmal sac, the vaso-occlusive device may then be placed into an expanded configuration, filling and occluding the aneurysmal sac. The vaso-occlusive device may deform or bend to allow more efficient and complete packing. The vaso-occlusive device is then released or “detached” from the distal end of the delivery assembly, and the delivery assembly is withdrawn back through the delivery catheter. Depending on the particular needs of the patient, one or more additional vaso-occlusive devices may be pushed through the delivery catheter and released into the same aneurysmal sac until the aneurysmal sac is completely filled with vaso-occlusive devices.

Notably, it is desirable that the vaso-occlusive devices that are delivered into an aneurysmal sac be as long as possible. That is, small (short) vaso-occlusive devices are less desirable, since delivery of such small vaso-occlusive devices into an aneurysmal sac may require a longer and more involved procedure. For example, a 7 mm diameter neurological aneurysmal sac may typically be filled with five to seven individual vaso-occlusive coils, resulting in a longer and more complicated procedure than if the number of devices was reduced. Thus, it is important that the lengths of vaso-occlusive devices be increased as much as possible to reduce the number of such vaso-occlusive devices needed to treat an aneurysm, and thus, thereby reducing the time and complexity of the procedure.

Fluoroscopy is typically used to visualize vaso-occlusive devices during delivery into an aneurysmal sac, while magnetic resonance imaging (MRI) is typically used to visualize the treatment site post-procedure (e.g., a few weeks after initial treatment of the aneurysm) to ensure that the aneurysmal sac is properly occluded. As such, it is important that vaso-occlusive devices, such as vaso-occlusive coils, be constructed in a manner that enables their radiopacity during treatment of the aneurysm, while minimizing their magnetic susceptibility, such that any visualization obscuring artifacts created during the post-procedure MRI is minimized (i.e., being MRI-compatible). It is also paramount that such vaso-occlusive devices be “soft” (i.e., be laterally flexible or conformable), and thus atraumatic, to prevent rupturing of the delicate tissues of the aneurysm.

One highly desirable means of delivering a vaso-occlusive device (such as a vaso-occlusive coil) into an aneurysmal sac employs an electrolytic detachment procedure, such as that described in U.S. Pat. No. 5,122,136, which is expressly incorporated herein by reference. After loading an electrically conductive delivery wire (e.g., composed of stainless steel (e.g., SS316)) with an attached vaso-occlusive coil within the delivery catheter and distally advancing the delivery wire to insert the vaso-occlusive coil into the aneurysmal sac, such electrolytic detachment procedure involves severing the vaso-occlusive coil from the distal end of delivery wire by the application of a small electric current through the delivery wire to an electrolytically severable joint between the vaso-occlusive coil and the distal end of the delivery wire that is exposed to the blood within the blood vessel. In a typical embodiment, the electrically conductive delivery wire is coated with an electrically insulative material (e.g., a polyimide-coated stainless steel (e.g., SS316). However, a small section (e.g., less than 0.15 inches in length) of the delivery wire just proximal to the vaso-occlusive device has not electrically insulative coating, thereby forming the electrolytically severable joint. Because, the electrolytically severable joint is not electrically insulated, it is more susceptible to electrolytic dissolution in blood than the portion of the delivery wire covered with the electrically insulative material and the vaso-occlusive device. Thus, the electrolytically severable joint will substantially or completely dissolve, thereby releasing the vaso-occlusive device into the aneurysmal sac.

It is important that the electrical resistivity of the electrolytically severable joint to be as low and electrochemical potential of the electrolytically severable joint be as negative as possible in order to maximize the speed of the electrolysis, and thus, electrolytic detach performance. Furthermore, because a portion of the electrolytically severable joint typically remains with the vaso-occlusive device after electrolytic detachment of the vaso-occlusive device from the delivery wire, it is important that, like the vaso-occlusive device itself, the electrolytically severable joint be constructed in a manner that minimizes any visualization obscuring artifacts created during the post-procedure MRI.

Notably, the desirability to increase the length of a vaso-occlusive device, while minimizing the lumen size of the delivery catheter, enabling the vaso-occlusive device with the necessary radiopacity during treatment of the aneurysm, minimizing any visualization obscuring artifacts created during the post-procedure MRI, providing the vaso-occlusive device with enough softness to prevent tissue trauma, and providing the electrolytically severable joint with minimum electrical resistivity and negative electrochemical potential are countervailing factors that present challenges to the material composition of the vaso-occlusive device, and more relevant to the present inventions, the material composition of the electrolytically severable joint.

In particular, increasing the length of a vaso-occlusive device necessarily increases the friction of such vaso-occlusive device and/or the lumen size of the delivery catheter. Thus, to maintain the relatively small lumen size, and thus outer diameter, of the delivery catheter, the columnar strength (buckling resistance) of the vaso-occlusive device, as well as the unsupported electrolytically severable joint, must be increased (i.e., the Young's modulus and mechanical strength increased) to ensure that the vaso-occlusive device can be delivered into the aneurysmal sac. Materials that enable a relatively long vaso-occlusive device to be delivered through a relatively small diameter delivery catheter, while satisfying the other countervailing requirements, including radiopacity (for the vaso-occlusive device) and MRI-compatibility requirements (for both the vaso-occlusive device and electrolytically severable joint), softness (for the vaso-occlusive device), low electrical resistivity and negative electrochemical potential (for the electrolytically severable joint), as well as being biocompatible, are very limited.

One advantageous embodiment of a vaso-occlusive device, disclosed in U.S. patent application Ser. No. 16/208,860, entitled “Vaso-Occlusive Device,” which is expressly incorporated herein by reference, is composed of a gold-platinum (AuPt) alloy. Such vaso-occlusive device may be relatively long, while also allowing the diameter of the delivery catheter to remain relatively small, as well as providing the necessary radiopacity during treatment of the aneurysm, minimizing any visualization obscuring artifacts created during the post-procedure MRI, and providing the necessary softness to prevent tissue trauma.

However, the columnar strength of present electrolytically severable joints, such as those composed of SS316, are limited, thereby at least partially negating the increased length in the vaso-occlusive device afforded by the use of a AuPt alloy in the construction of the vaso-occlusive device. Furthermore, the remnants of such electrolytically severable joints left on vaso-occlusive devices after electrolytic detachment have a relatively high magnetic susceptibility, such that they will cast a residual MR artifact, thereby negating some of the MR artifact reducing benefit of MRI-compatible vaso-occlusive devices, and possibly making post-procedure MRIs challenging even when MRI-compatible vaso-occlusive devices (such as those composed of an AuPt alloy) are employed.

There, thus, remains a need to provide an electrolytically severable joint for a relatively long vaso-occlusive device that has a relatively high columnar strength, while having a relatively low magnetic susceptibility, a relatively low electrical resistivity, and a negative electrochemical potential.

### SUMMARY

In accordance with the present inventions, a vaso-occlusive assembly comprises a vaso-occlusive device (e.g., a vaso-occlusive coil) configured for implantation in an aneurysmal sac. The vaso-occlusive device has a delivery configuration when restrained within a delivery catheter and has a deployed configuration when released from the delivery catheter into the aneurysmal sac. The vaso-occlusive device may have a suitable length, e.g., greater than 5 cm. The delivery catheter may have an inner lumen (e.g., having a diameter less than 0.020 inches) in which the vaso-occlusive device is retrained. The vaso-occlusive assembly further comprises a delivery wire having a distal end to which the vaso-occlusive device is affixed. The delivery wire has an electrolytically severable joint proximal to the vaso-occlusive device. In one embodiment, a vaso-occlusive treatment system comprises the vaso-occlusive assembly, the delivery catheter, and an electrolytic detachment device to which a proximal end of the delivery wire of the vaso-occlusive assembly is configured for being electrically coupled. The electrolytic detachment device is configured for delivering electrical current to the electrolytically severable joint of the vaso-occlusive assembly while the vaso-occlusive device is disposed within aneurysmal sac, such that the vaso-occlusive device electrolytically detaches from the distal end of the delivery wire.

In accordance with a first aspect of the present inventions, the electrolytically severable joint is composed of molybdenum (Mo) or an alloy containing Mo greater than 20% by weight. In one embodiment, the alloy contains Mo greater than 40% by weight. In another embodiment, the electrolytically severable joint may be composed of a Mo alloy, such as, e.g., molybdenum (Mo)-rhenium (Re), molybdenum (Mo)-tungsten (W), molybdenum (Mo)-rhodium (Rh), molybdenum (Mo)-Iridium (Ir), molybdenum (Mo)-platinum (Pt), molybdenum (Mo)-palladium (Pd), molybdenum (Mo)-gold (Au), molybdenum (Mo)-Tantalum (Ta), molybdenum (Mo)-niobium (Nb), molybdenum (Mo)-zirconium (Zr), molybdenum (Mo)-cerium (Ce), hafnium (Hf), or any combination thereof. In one specific embodiment, the Mo alloy is Mo—Re. In this case, the Re contained in the Mo alloy may be greater than 20% by weight, e.g., 47.5% by weight. In other embodiments, the alloy may have a Young's modulus greater than 35 Msi, a mechanical strength greater than 400 Ksi, a magnetic susceptibility less than 300, an electrical resistivity less than 100μσ·cm, and/or an electrochemical potential less than −0.1 V.

In accordance with a second aspect of the present inventions, the electrolytically severable joint is composed of an alloy containing molybdenum (Mo), and having a Young's modulus greater than 35 Msi. In one embodiment, the Young's modulus of the alloy is greater than 40 Msi, and in another embodiment, the Young's modulus of the alloy is greater than 45 Msi. In still another embodiment, the alloy contains Mo greater than 20% by weight, and even greater than 40% by weight. In yet another embodiment, the electrolytically severable joint may be composed of a Mo alloy, such as, e.g., molybdenum (Mo)-rhenium (Re), molybdenum (Mo)-tungsten (W), molybdenum (Mo)-rhodium (Rh), molybdenum (Mo)-Iridium (Ir), molybdenum (Mo)-platinum (Pt), molybdenum (Mo)-palladium (Pd), molybdenum (Mo)-gold (Au), molybdenum (Mo)-Tantalum (Ta), molybdenum (Mo)-niobium (Nb), molybdenum (Mo)-zirconium (Zr), molybdenum (Mo)-cerium (Ce), hafnium (Hf), or any combination thereof. In one specific embodiment, the Mo alloy is Mo—Re. In this case, the Re contained in the Mo alloy may be greater than 20% by weight, e.g., 47.5% by weight. In other embodiments, the alloy may have a mechanical strength greater than 400 Ksi, a magnetic susceptibility less than 300, an electrical resistivity less than 100μσ·cm, and/or an electrochemical potential less than −0.1 V.

In accordance with a third aspect of the present inventions, the electrolytically severable joint is composed of an alloy containing molybdenum (Mo), and having a mechanical strength greater than 400 Ksi. In one embodiment, the mechanical strength of the alloy is greater than 450 Ksi, and in another embodiment, the mechanical strength of the alloy is greater than 500 Ksi. In still another embodiment, the alloy contains Mo greater than 20% by weight, and even greater than 40% by weight. In yet another embodiment, the electrolytically severable joint may be composed of a Mo alloy, such as, e.g., molybdenum (Mo)-rhenium (Re), molybdenum (Mo)-tungsten (W), molybdenum (Mo)-rhodium (Rh), molybdenum (Mo)-Iridium (Ir), molybdenum (Mo)-platinum (Pt), molybdenum (Mo)-palladium (Pd), molybdenum (Mo)-gold (Au), molybdenum (Mo)-Tantalum (Ta), molybdenum (Mo)-niobium (Nb), molybdenum (Mo)-zirconium (Zr), molybdenum (Mo)-cerium (Ce), hafnium (Hf), or any combination thereof. In one specific embodiment, the Mo alloy is Mo—Re. In this case, the Re contained in the Mo alloy may be greater than 20% by weight, e.g., 47.5% by weight. In other embodiments, the alloy may have a Young's modulus greater than 35 Msi, a magnetic susceptibility less than 300, an electrical resistivity less than 100 μσ·cm, and/or an electrochemical potential less than −0.1 V.

In accordance with a fourth aspect of the present inventions, the electrolytically severable joint is composed of an alloy containing molybdenum (Mo), and having a magnetic susceptibility less than 300. In one embodiment, the magnetic susceptibility of the alloy is less than 200, and in another embodiment, the magnetic susceptibility of the alloy is less than 150. In still another embodiment, the alloy contains Mo greater than 20% by weight, and even greater than 40% by weight. In yet another embodiment, the electrolytically severable joint may be composed of a Mo alloy, such as, e.g., molybdenum (Mo)-rhenium (Re), molybdenum (Mo)-tungsten (W), molybdenum (Mo)-rhodium (Rh), molybdenum (Mo)-Iridium (Ir), molybdenum (Mo)-platinum (Pt), molybdenum (Mo)-palladium (Pd), molybdenum (Mo)-gold (Au), molybdenum (Mo)-Tantalum (Ta), molybdenum (Mo)-niobium (Nb), molybdenum (Mo)-zirconium (Zr), molybdenum (Mo)-cerium (Ce), hafnium (Hf), or any combination thereof. In one specific embodiment, the Mo alloy is Mo—Re. In this case, the Re contained in the Mo alloy may be greater than 20% by weight, e.g., 47.5% by weight. In other embodiments, the alloy may have a Young's modulus greater than 35 Msi, a mechanical strength greater than 400 Ksi, an electrical resistivity less than 100μσ·cm, and/or an electrochemical potential less than −0.1 V.

In accordance with a fifth aspect of the present inventions, the electrolytically severable joint is composed of an alloy having a Young's modulus greater than 35 Msi, a mechanical strength greater than 400 Ksi, and a magnetic susceptibility less than 300. In one embodiment, the Young's modulus of the alloy is greater than 40 Msi, the mechanical strength of the alloy is greater than 450 Ksi, and the magnetic susceptibility of the alloy is less than 200. In another embodiment, the Young's modulus of the alloy is greater than 45 Msi, the mechanical strength of the alloy is greater than 500 Ksi, and the magnetic susceptibility of the alloy is less than 150. In still another embodiment, the alloy contains Mo. For example, the alloy may contain Mo greater than 20% by weight, and even greater than 40% by weight. In yet another embodiment, the alloy may be a Mo alloy, such as, e.g., molybdenum (Mo)-rhenium (Re), molybdenum (Mo)-tungsten (W), molybdenum (Mo)-rhodium (Rh), molybdenum (Mo)-Iridium (Ir), molybdenum (Mo)-platinum (Pt), molybdenum (Mo)-palladium (Pd), molybdenum (Mo)-gold (Au), molybdenum (Mo)-Tantalum (Ta), molybdenum (Mo)-niobium (Nb), molybdenum (Mo)-zirconium (Zr), molybdenum (Mo)-cerium (Ce), hafnium (Hf), or any combination thereof. In one specific embodiment, the Mo alloy is Mo—Re. In this case, the Re contained in the Mo alloy may be greater than 20% by weight, e.g., 47.5% by weight. In other embodiments, the alloy may have an electrical resistivity less than 100μσ·cm and/or an electrochemical potential less than −0.1 V.

Other and further aspects and features of embodiments will become apparent from the ensuing detailed description in view of the accompanying figures.

### BRIEF DESCRIPTION OF THE DRAWINGS

The drawings illustrate the design and utility of preferred embodiments of the disclosed inventions, in which similar elements are referred to by common reference numerals. It should be noted that the figures are not drawn to scale and that elements of similar structures or functions are represented by like reference numerals throughout the figures. It should also be noted that the figures are only intended to facilitate the description of the embodiments. They are not intended as an exhaustive description of the invention or as a limitation on the scope of the invention, which is defined only by the appended claims and their equivalents. In addition, an illustrated embodiment of the disclosed inventions needs not have all the aspects or advantages shown. Further, an aspect or an advantage described in conjunction with a particular embodiment of the disclosed inventions is not necessarily limited to that embodiment and can be practiced in any other embodiments even if not so illustrated.

In order to better appreciate how the above-recited and other advantages and objects of the disclosed inventions are obtained, a more particular description of the disclosed inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is a plan view of a vaso-occlusive treatment system constructed in accordance with one embodiment of the disclosed inventions, particularly showing a vaso-occlusive device in a delivery configuration;

FIG. 2 is a plan view of the vaso-occlusive treatment system of FIG. 1, particularly showing the vaso-occlusive device in a deployed configuration;

FIG. 3 is a plan view of a vaso-occlusive structure of the vaso-occlusive treatment system of FIG. 1, deployed within an aneurysmal sac; and

FIG. 4 is a close-up, partially-cutaway, cross-sectional view of an electrolytically severable joint of a vaso-occlusive assembly of the vaso-occlusive treatment system of FIG. 1.

### DETAILED DESCRIPTION OF THE ILLUSTRATED EMBODIMENTS

The present disclosure is directed to a vaso-occlusive assembly used in a vaso-occlusive treatment system that delivers vaso-occlusive devices (e.g., vaso-occlusive coils) within the vasculature of a patient (e.g., within an aneurysmal sac) via an electrolytic detachment procedure. The vaso-occlusive device described herein comprises a delivery wire having an electrolytically severable joint composed of molybdenum (Mo) or an alloy thereof that has a columnar strength high enough to facilitate the delivery of a relatively long vaso-occlusive device with a higher push friction through a relatively small diameter delivery catheter (i.e., a microcatheter), while having a magnetic susceptibility low enough to minimize visualization obscuring artifacts created during the post-procedure MRI (i.e., MRI compatible), and having a negative electrochemical potential and an electrical resistivity that is low enough to maximize electrolytic detach performance.

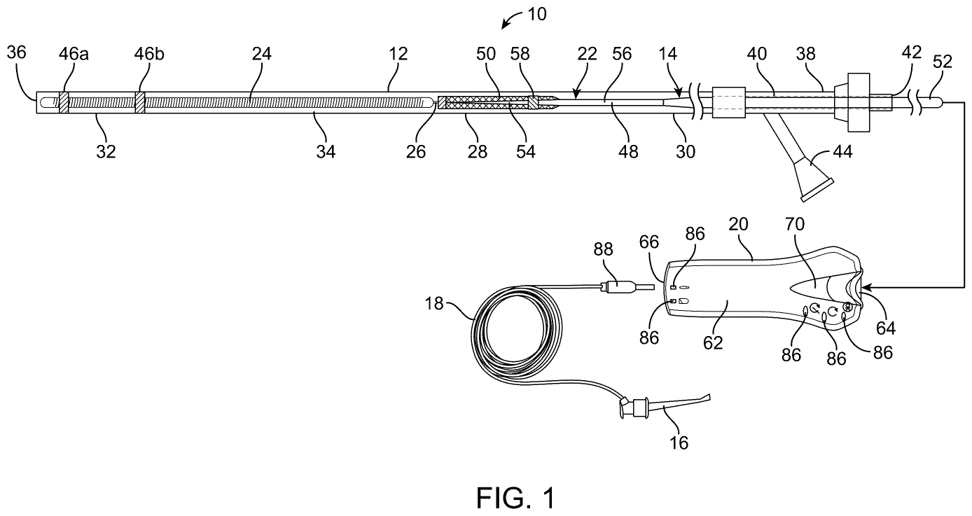

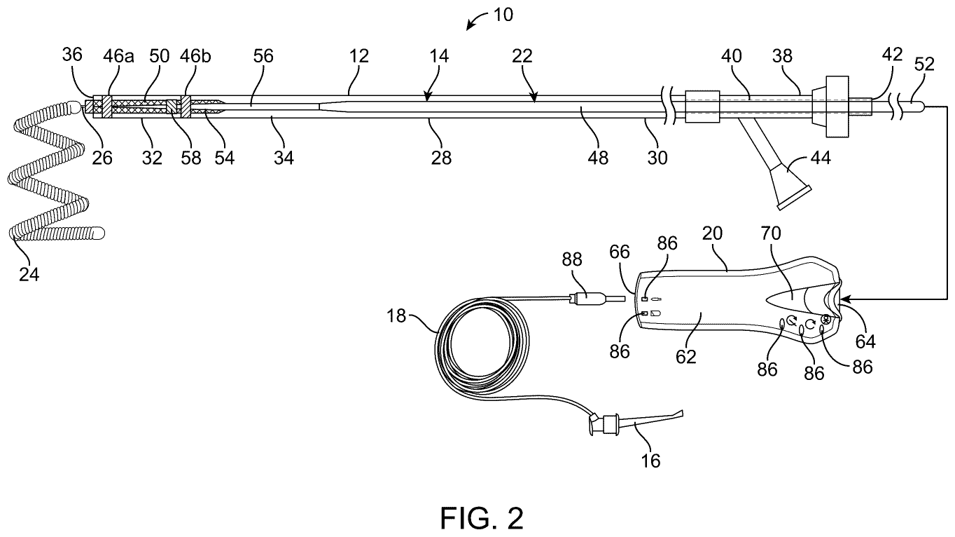

Referring to FIGS. 1-2, one embodiment of a vaso-occlusive treatment system 10 constructed in accordance with the disclosed inventions will now be described. The vaso-occlusive treatment system 10 comprises a delivery catheter 12, a vaso-occlusive assembly 14 slidably disposed within the delivery catheter 12, a ground electrode 16 configured for being placed in contact with a patient, an electrical cable 18 configured for being removably affixed to the ground electrode 16, and an electrical power source in the form of an electrolytic detachment device 20 to which the vaso-occlusive assembly 14 is removably affixed, and to which the ground electrode 16 is removably affixed via the electrical cable 18. As will be discussed in further detail below, the vaso-occlusive assembly 14 comprises a delivery wire 22 and a vaso-occlusive device 24 detachably coupled to the delivery wire 22 via an electrolytically severable joint 26.

The delivery catheter 12 has a tubular configuration, and can, e.g., take the form of a delivery catheter, a sheath, or the like. The delivery catheter 12 comprises an elongate sheath body 28 having a proximal portion 30 and a distal portion 32, and an inner lumen 34 (shown partially in phantom) extending through the sheath body 28 between the proximal portion 30 and the distal portion 32, and in which the vaso-occlusive assembly 14 is housed. The inner lumen 34 preferably has a relatively small diameter, e.g., less than 0.03 inches, and preferably less than 0.02 inches, such that the outer diameter of sheath body 28 may be minimized. In alternative embodiments, the delivery catheter 12 may have multiple inner lumens (not shown) in which multiple vaso-occlusive assemblies 14 may be housed.

The free end of the proximal portion 30 of the sheath body 28 remains outside of the patient and accessible to an operator (e.g., clinician or physician), while the remainder of the sheath body 28, including the distal portion 32, is sized and dimensioned to reach remote locations of the vasculature of the patient. The sheath body 28 has a suitable length for accessing a target tissue site within the patient from a vascular access point. The target tissue site depends on the medical procedure for which the delivery catheter 12 is used. In one embodiment, the outer diameter of the sheath body 28 may be uniform along the length of the sheath body 28. In another embodiment, the outer diameter of the sheath body 28 may taper in either a gradual fashion or a step-wise fashion from a first outer diameter of the proximal portion 30 to a second outer diameter at the distal portion 32 to facilitate navigation in tortuous vasculature. Although depicted as having a generally round cross-sectional shape, it can be appreciated that the sheath body 28 can include other cross-sectional shapes or combinations of shapes, e.g., oval, rectangular, triangular, polygonal, and the like.

The delivery catheter 12 may include one or more, or a plurality of regions along its length having different configurations and/or characteristics. For example, the distal portion 32 of the sheath body 28 may have an outer diameter less than the outer diameter of the proximal portion 30 of the sheath body 28 to reduce the profile of the distal portion 32 and facilitate navigation in tortuous vasculature. Furthermore, the distal portion 32 may be more flexible than the proximal portion 30. Generally, the proximal portion 30 may be formed from material that is stiffer than the distal portion 32 of the sheath body 28, so that the proximal portion 30 has sufficient pushability to advance through the patient's vascular system, while the distal portion 32 may be formed of a more flexible material so that the distal portion 32 may remain flexible and track more easily over a guidewire to access remote locations in tortuous regions of the vasculature. The sheath body 28 may be composed of suitable polymeric materials, metals and/or alloys, such as polyethylene, stainless steel or other suitable biocompatible materials or combinations thereof. In some instances, the proximal portion 30 may include a reinforcement layer, such a braided layer or coiled layer to enhance the pushability of the sheath body 28. The sheath body 28 may include a transition region between the proximal portion 30 and the distal portion 32.

The delivery catheter 12 comprises a distal port 36 in communication with the inner lumen 34 of the delivery catheter 12 and from which the vaso-occlusive device 24 is deployed. The delivery catheter 12 further comprises a proximal adapter 38 affixed to the proximal portion 30 of the sheath body 28 using suitable means, e.g., adhesive, welding, etc. The proximal adapter 38 comprises a central bore 40 (shown in phantom) in communication with the lumen 34 of the delivery catheter 12. The central bore 40 terminates in a proximal port 42 for allowing loading of the vaso-occlusive assembly 14 into the delivery catheter 12. The proximal adapter 38 further comprises a side port 44 in fluid communication with the central bore 40 for introducing fluids into the inner lumen 34 of the delivery catheter 12, e.g., to vaso-occlusive assembly 14, to introduce contrast into the vasculature of the patient, and/or to introduce saline into the vasculature of the patient, e.g., to flush out contrast prior to electrolytic detachment and delivery of the vaso-occlusive device 24 into the vasculature of the patient.

The delivery catheter 12 further comprises one or more radiopaque marker bands 46 (in this case, two distal and proximal bands 46a, 46b) disposed on the distal portion 32 of the delivery catheter 12 proximate the distal port 36, which can be identified using medical imaging technology (e.g., fluoroscopy). The distal band 46a may be used to locate the distal tip of the delivery catheter 12 within the patient's vasculature system, while the proximal band 46b may be used to locate the delivery catheter 12 relative to the partially or fully deployed vaso-occlusive device 24, such that the delivery catheter 12 and delivery wire 22 may be longitudinally aligned to ensure that the electrolytically severable joint 26 is located just distal to the distal port 36 of the delivery catheter 12 in contact with bodily fluids in the vasculature of the patient to facilitate electrolytic detachment of the vaso-occlusive device 24 from the delivery wire 22, as will be discussed in further detail below. The radiopaque marker bands 46 may be composed of a suitable radiopaque material, e.g., gold, platinum, palladium, tantalum, tungsten alloy, polymer material loaded with a radiopaque filler, and the like.

In the illustrated embodiment, the delivery wire 22 is monopolar in nature in that it is only capable of delivering electrical current from the electrolytic detachment device 20 to the electrolytically severable joint 26, which electrical current is then returned to electrolytic detachment device 20 via the ground electrode 16 placed in contact with the patient. In this case, the delivery wire 22 may comprise only a power terminal (not shown) for delivering electrical current from the electrolytic detachment device 20 to the electrolytically severable joint 26. In an alternative embodiment, the delivery wire 22 may be bipolar in nature in that it is capable of conducting electrical current to and from the electrolytically severable joint 26. In this case, the delivery wire 22 may comprise bipolar terminals (not shown) disposed on the proximal portion 52 of the core wire 48 delivering electrical current from the electrolytic detachment device 20 to the electrolytically severable joint 26, and returning electrical current from the electrolytically severable joint 26 to the electrolytic detachment device 20.

In general, the vaso-occlusive device 24 may be inserted into the patient by first inserting (e.g., minimally invasively) the delivery catheter 12 into the patient's vasculature to reach the aneurysm site. If the delivery catheter 12 is used to access vasculature in the brain of a patient from a femoral artery access point at the groin of the patient, the overall length of the sheath body 28 may be 125 cm-200 cm. Furthermore, the diameter of the delivery catheter 12 is made as small as possible. For example, the distal portion 32 of the sheath body 28 may have a relatively small outer diameter (e.g., less than 3F) and a relatively small inner diameter (i.e., size of lumen 28) (e.g., less than 0.020″, such as, e.g., between 0.015″ and 0.025″, and preferably between 0.015″ and 0.018″).

The delivery catheter 12 may be used in an “over-the-wire” configuration, wherein the delivery catheter 12 is introduced into the patient over a guidewire that has been previously introduced, and the delivery catheter 12 extends over the entire length of the guidewire (not shown). Alternatively, the delivery catheter 12 may be used in a “rapid-exchange” configuration, where a guidewire extends through only a distal portion of the delivery catheter 12 from a guidewire port (not shown). In other alternative embodiments, the delivery catheter 12 may be introduced into the patient after a guidewire has been withdrawn, leaving a sheath or access catheter distal portion at the target site for the delivery catheter 12 to navigate through the vasculature of the patient within the sheath or access catheter.

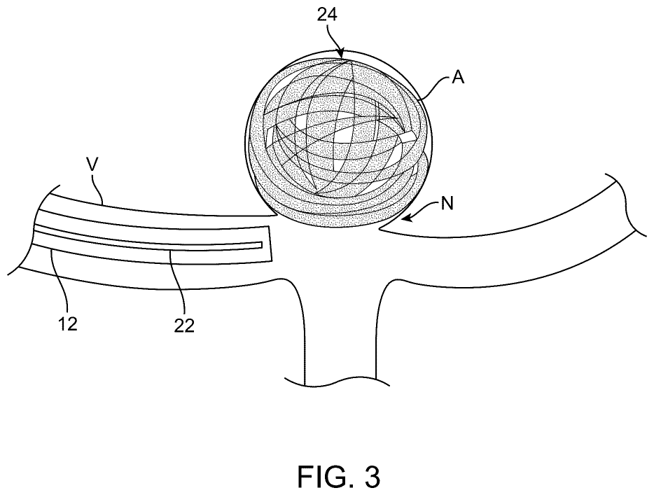

At the aneurysm site, the vaso-occlusive device 24 may be pushed distally out of the delivery catheter 12 residing in the parent vessel V through the aneurysmal neck N and into an aneurysmal sac A via the delivery wire 22, as illustrated in FIG. 3. After being extruded from the delivery catheter 12, the vaso-occlusive device 24 may self-expand into a pre-set configuration as described below. Once the vaso-occlusive device 24 is inserted into the aneurysmal sac A, the vaso-occlusive device 24 may be electrolytically decoupled from the delivery wire 22, as will be described in further detail below. A sufficient number of vaso-occlusive devices 24 may be delivered to fill and occlude the aneurysmal sac A. The vaso-occlusive device 24 may also be removed or withdrawn, and collapsed back into the delivery catheter 12 by proximally withdrawing the vaso-occlusive device 24 via the delivery wire 22 prior to electrolytic detachment thereof.

In general, the vaso-occlusive device 24 has a delivery configuration when restrained within the delivery catheter 12 (FIG. 1) and a deployed configuration that conforms to the interior shape of an aneurysmal sac A when deployed from the delivery catheter 12 (FIG. 2) into the aneurysmal sac A. The vaso-occlusive device 24 may be pre-biased to form a cylinder, a cone, or other desired envelope. The vaso-occlusive device 24 may be extremely soft and its overall shape easily deformed. In the illustrated embodiment, the vaso-occlusive device 24 is shown as a helical coil formed of a wire having a suitable diameter, e.g., 1-6 mils. The diameter of the vaso-occlusive device 24, when in the delivery configuration, may be, e.g., 10-30 mils. The vaso-occlusive device 24 may have any suitable length desirable and appropriate for the site to be occluded, e.g., 1-60 cm. In alternative embodiments, the vaso-occlusive device 24 may take the form of a structure other than a coil, e.g., a braid. The vaso-occlusive device 24 may optionally be covered or connected with fibrous materials tied to the outside of the coil or braid. The vaso-occlusive device 24 may be composed of a suitable biocompatible and radio-opaque material, such as platinum, gold, tungsten, iridium, or alloys thereof or other metals. In one advantageous embodiment, the vaso-occlusive device 24 is composed of a gold-platinum (AuPt) alloy (e.g., AuPt34), which has been demonstrated to have good columnar strength, good radiopacity, good MRI compatibility, as disclosed in U.S. patent application Ser. No. 16/208,860, entitled “Vaso-Occlusive Device,” which has previously been expressly incorporated herein by reference. Thus, the length of the vaso-occlusive device 24 may be relatively long (e.g., greater than 5 cm, e.g., between 5 cm and 45 cm). In the illustrated embodiment, the vaso-occlusive device 24 has an end cap or tip that prevents punctures of the aneurysmal sac A when delivered therein.

The delivery wire 22 may be a coil, wire, tendon, or the like (e.g., a conventional guidewire, torqueable cable tube, or a hypotube), having a sufficient columnar strength to permit pushing of the vaso-occlusive device 24 into the aneurysmal sac A. The delivery wire 22 may have a suitable outer diameter, e.g., 10-30 mils, and a suitable length, e.g., 50-300 cm. The material used to construct the delivery wire 22 is chosen to impart varying flexibility and stiffness characteristics to different portions of the delivery wire 22. For example, the delivery wire 22 may be formed of different materials along its length, for example materials having different moduli of elasticity, resulting in a difference in flexibility.

In the illustrated embodiment, the delivery wire 22 generally comprises a core wire 48 composed of an electrically conductive material and a sleeve 50 composed of an electrically insulative material, such as, e.g., polytetrafluoroethylene, polyurethane, polyethylene, polypropylene, or other suitable polymeric material. The core wire 48 has a proximal portion 52 that extends proximal from the proximal portion 30 of the delivery catheter 12 for manipulation by the physician, a distal portion 54 to which the vaso-occlusive device 24 is attached, and a medial portion 56 disposed between the proximal portion 52 and the distal portion 54. The proximal portion 52 of the core wire 48 is enlarged to ergonomically facilitate manipulation of the delivery wire 22 by the physician. The proximal portion 52 of the core wire 48 distally tapers downward to the medial portion 56. The distal portion 54 of the core wire 48 extends from the medial portion 56 and distally tapers further downward to provide flexibility to the distal end of the delivery wire 22. The delivery wire 22 may comprise a coil (not shown) affixed around the distal portion 54 of the core wire 48 to provide some columnar strength to the distal end of the delivery wire 22, while not detrimentally affecting the flexibility of the tapered distal portion 54 of the core wire 48. The sleeve 50 is disposed over the distal portion 54 of the core wire 48, and as discussed in further detail, serves to electrically isolate the portion of the distal portion 54 of the core wire 48 that is proximal to the electrolytically severable joint 26 of the delivery wire 22, from the blood in the vasculature of the patient. The delivery wire 22 further comprises a radiopaque marker band 58 disposed over the sleeve 50, which can be identified using medical imaging technology (e.g., fluoroscopy). The marker band 58 may be used to locate the delivery catheter 12 relative to the partially or fully deployed vaso-occlusive device 24 (by aligning it relative to the proximal marker 46b of the delivery catheter 12), such that the delivery catheter 12 and delivery wire 22 may be longitudinally aligned to ensure that the electrolytically severable joint 26 is located just distal to the distal port 36 of the delivery catheter 12 in contact with bodily fluids in the vasculature of the patient to facilitate electrolytic detachment of the vaso-occlusive device 24 from the delivery wire 22, as will be discussed in further detail below. The radiopaque marker band 58 may be composed of a suitable radiopaque material, e.g., gold, platinum, palladium, tantalum, tungsten alloy, polymer material loaded with a radiopaque filler, and the like.

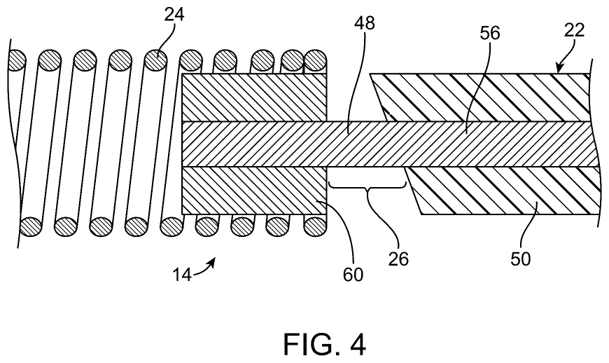

Referring further to FIG. 4, the vaso-occlusive device 24 is affixed to the distal portion 56 of the core wire 48 via an electrolytically-resistant bushing 60. The electrolytically severable joint 26 takes the form of an electrolytically degradable segment for electrolytically decoupling the vaso-occlusive device 24 from the delivery wire 22, and is located on the core wire 48 between the electrically insulative sleeve 50 and the vaso-occlusive device 24. Thus, when electrical current is supplied to the core wire 46, the electrical current flows to the electrolytically severable joint 26. However, the electrolytically severable joint 26 is not electrically insulated, and is, thus, more susceptible to electrolytic dissolution in blood than the portion of the core wire 48 covered with the electrically insulative sleeve 50 and the vaso-occlusive device 24. Thus, the electrolytically severable joint 26 will dissolve when the electrical current is applied to the core wire 48, thereby releasing the vaso-occlusive device 24. Preferably, the length of the electrolytically severable joint 26 is not much greater than the diameter of the electrolytically severable joint 26. For example, the electrolytically joint 26 may be as short as 0.001 inches, and typically no longer than 0.010 inches in length.

In the illustrated embodiment, the ground electrode 16 takes the form of a metallic clip that is configured for being removably attached to a hypodermic needle (not shown) that has been percutaneously inserted into the patient, e.g., into the thigh or groin of the patient, such that the ground electrode 16 is electrically coupled to the patient, thereby completing an electrical circuit that electrically couples the vaso-occlusive assembly 14 to the ground electrode 16 through the electrically conductive patient.

The electrolytic detachment device 20 may be operated by the physician to perform an electrolytic detachment procedure. The electrolytic detachment device 20 comprises an outer casing 62; a power terminal 64 to which the core wire 48 is electrically coupled; a ground terminal 66 to which the ground electrode 16 is electrically coupled via the electrical cable 18; electronic componentry (not shown) contained within the outer casing 62 for delivering electrical current to electrolytic severable joint 26 of the vaso-occlusive assembly 14 in a controlled manner; and an electrolytic detachment actuator 70 affixed to the outer casing 62 for manually initiating the flow of electrical current from the electrolytic detachment device 20 to the electrolytic severable joint 26 of the vaso-occlusive assembly 14.

The outer casing 62 is composed of a suitable material, e.g., Acrylonitrile Butadiene Styrene (ABS) or polycarbonate, is a shaped and sized to be ergonomically held by a physician with one hand. In the illustrated embodiment, the power terminal 64 takes the form of a port (e.g., a funnel) in which the proximal portion 52 of the core wire 48 may be alternately inserted and removed, while the ground terminal 66 takes the form of a port in which a corresponding plug 88 of the electrical cable 18 may be alternately inserted and removed. The electronic componentry is configured for delivering electrical current to the electrolytic severable joint 26 of the vaso-occlusive assembly 14 during one or more electrolytic detachment cycles until the vaso-occlusive device 24 electrolytically detaches from the delivery wire 22, as well as detecting electrolytic detachment of the vaso-occlusive device 24 from the delivery wire 22, and reporting to the physician of various events that occur during the electrolytic detachment procedure via various indicators 86. Further details on such electronic componentry are set forth in U.S. Provisional Application Ser. No. 63/486,183, entitled “Vaso-Occlusive Electrolytic Detachment Detection,” which is expressly incorporated herein by reference.

In the illustrated embodiment, the electrolytic detachment actuator 70 takes the form of a push button, which can be depressed to manually command the electrolytic detachment device 20 to perform an electrolytic detachment cycle (i.e., a time period during which electrical current is delivered from the electronic componentry (not shown) to the electrolytic severable joint 26 of the vaso-occlusive assembly 14). A single actuation of the push button 70 (that is, quickly depressed and then released) initiates an electrolytic detachment cycle. The push button 70 may be actuated multiple times to initiate a series of electrolytic detachment cycles. That is, the push button 70 may be actuated to initiate a first electrolytic detachment cycle, then after the first electrolytic detachment cycle terminates, the push button 70 may be actuated again to initiate a second electrolytic detachment cycle, and then after the second electrolytic detachment cycle terminates, the push button 70 may be actuated again to initiate a third electrolytic detachment cycle, and so forth.

Significantly, it is important that the electrolytically severable joint 26 has the necessary columnar strength to facilitate the delivery of the relatively long vaso-occlusive device 24 (e.g., greater than 5 cm) through the relatively small diameter delivery catheter 12 (e.g., one having a lumen 34 less than 0.03 inches in diameter). Furthermore, since a distal portion of the electrolytically severable joint 26 (i.e., the distal portion thereof) will likely remain with the vaso-occlusive device 24 after electrolytic detachment of the vaso-occlusive device 24 from the delivery wire 22, it is important that at least the distal portion of the electrolytically severable joint 26 be MRI-compatible. Furthermore, it is important that the electrolytically severable joint 26 have good electrolytic detach performance.

The inventors have discovered that molybdenum (Mo) and certain metal alloys thereof, and any combination thereof, enable the electrolytically severable joint 26, given a relatively small diameter, to have the columnar strength high enough to facilitate the delivery of the relatively long vaso-occlusive device 24 through the relatively small diameter delivery catheter 12, while being MRI compatible to minimize visualization obscuring artifacts created during the post-procedure MRI, and while having a relatively high electrolytic detach performance. Thus, at least the distal portion electrolytically severable joint 26, and for ease of manufacture, preferably the entirety of the electrolytically severable joint 26, and in some embodiments the entirety of the core wire 48, may be composed of Mo or certain alloys containing Mo.

For example, compared to SS316, which has a Young's modulus of approximately 29 million pounds per square inch (Msi) and a mechanical strength in the range of 270-330 thousand pounds per square inch (Ksi), molybdenum has a Young's modulus of 47.1 Msi and a mechanical strength in the range of 400-500 Ksi.

Compared to SS316, which has a magnetic susceptibility in the range of 3570-6700, the magnetic susceptibility of Mo is 123. Furthermore, compared to SS316, which has an electrical resistivity of 74 micro-siemens per centimeter (μσ·cm) and an electrochemical potential of less than −0.5 volts (V), Mo has a significantly lower electrical resistivity of 5.5 micro-siemens per centimeter (μσ·cm), and an electrochemical potential of less than −0.2 volts (V). Thus, it should be appreciated that an electrolytically severable joint 26 composed of Mo has a significantly greater columnar strength (approximately 50% stronger and 62% stiffer), yet has a better MRI-compatibility and a similar electrolytic detach performance, than that of an electrolytically severable joint 26 composed of SS316.

Certain metals can be combined with Mo to yield alloys that can be used to construct an electrolytically severable joint 26 with additionally increased columnar strength, while still providing the necessarily MRI compatibility and electrolytic detach performance. For example, to enable the electrolytically severable joint 26 to have the necessary high columnar strength, the alloy used to construct the electrolytically severable joint 26 should have a Young's modulus greater than 35 million pounds per square inch (35 Msi) and a mechanical strength greater than 400 thousand pounds per square inch (400 Ksi); preferably, a Young's modulus greater than 40 Msi and a mechanical strength greater than 450 Ksi; and more preferably, a Young's modulus greater than 45 Msi and a mechanical strength greater than 500 Ksi. Furthermore, to enable the electrolytically severable joint 26 to have the necessary MRI compatibility, the alloy used to construct the electrolytically severable joint 26 should have a magnetic susceptibility less than 300, preferably less than 200, and more preferably less than 150. Furthermore, to enable the electrolytically severable joint 26 to have the necessary electrolytic detach performance, the alloy used to construct the electrolytically severable joint 26 should have an electrical resistivity less than 100 micro-siemen per centimeter per (μσ·cm), preferably less than 75μσ·cm, and more preferably less than 50μσ·cm, and an electrochemical potential of less than −0.1 volt (V). It is preferred that the percentage of Mo in weight in such alloys be greater than 20%, and preferably greater than 40%, to ensure that the resulting electrolytically severable joint 26 has the necessary MRI compatibility. The alloy may be, e.g., a Mo alloy, meaning that the Mo contained in the alloy is higher by weight than any other component in the alloy.

One particular Mo alloy (molybdenum with 47.5 percent rhenium by weight (Mo-47.5Re)) has a Young's modulus of 52.9 Msi, a mechanical strength in the range of 600-800 Ksi, a magnetic susceptibility of 110, an electrical resistivity of 22μσ·cm, and an electrochemical potential of less than −0.4 volt (V). Thus, it should be appreciated that an electrolytically severable joint 26 composed of Mo-47.5Re has an even greater columnar strength (more than 100% stronger and 82% stiffer), yet has a better MRI-compatibility and at least the same electrolytic detach performance, than that of an electrolytically severable joint 26 composed of SS316. Experimental studies using prototype bipolar delivery wires have shown that a 0.002″ diameter and a 0.002″ long electrolytically severable joint 26 composed of Mo-47.5Re has an average electrolytic detach time of 7.4 seconds—an electrolytic detach time that is similar to that of a 0.002″ electrolytically severable joint 26 composed of SS316. The electrolytic detach time of a 0.00175″ diameter electrolytic severable joint 26 composed of Mo-47.5Re is expected to be reduced to 5-6 seconds. While the Mo—Re alloy has been described as containing 47.5 percent rhenium by weight, the weight of rhenium in other Mo—Re alloys (or any other alloy containing Mo) may be, e.g., in the range of 20-60% by weight.

Other types of Mo alloys that may satisfy the foregoing ranges of Young's moduli, mechanical strengths, magnetic susceptibility, and electrical resistivity include molybdenum (Mo)-tungsten (W), molybdenum (Mo)-rhodium (Rh), molybdenum (Mo)-Iridium (Ir), molybdenum (Mo)-platinum (Pt), molybdenum (Mo)-palladium (Pd), molybdenum (Mo)-gold (Au), molybdenum (Mo)-Tantalum (Ta), molybdenum (Mo)-niobium (Nb), molybdenum (Mo)-zirconium (Zr), molybdenum (Mo)-cerium (Ce), hafnium (Hf). In contrast, other types of alloys, such as, e.g., cobalt Co-based alloys, which may not contain Mo (or may contain a relatively small percentage of Mo by weight), will have lower Young's moduli and mechanical strengths and/or relatively high magnetic susceptibilities, and will thus, be unsuitable at certain weight percentages.

Although particular embodiments have been shown and described herein, it will be understood by those skilled in the art that they are not intended to limit the disclosed inventions, and it will be obvious to those skilled in the art that various changes, permutations, and modifications may be made (e.g., the dimensions of various parts, combinations of parts) without departing from the scope of the disclosed inventions, which is to be defined only by the following claims and their equivalents. The specification and drawings are, accordingly, to be regarded in an illustrative rather than restrictive sense. The various embodiments shown and described herein are intended to cover alternatives, modifications, and equivalents of the disclosed inventions, which may be included within the scope of the appended claims.

What is claimed is:

1. A vaso-occlusive assembly, comprising:
a vaso-occlusive device configured for implantation in an aneurysmal sac, the vaso-occlusive device having a delivery configuration when restrained within a delivery catheter and having a deployed configuration when released from the delivery catheter into the aneurysmal sac; and
a delivery wire having a distal end to which the vaso-occlusive device is affixed, the delivery wire having an electrolytically severable joint proximal to the vaso-occlusive device, the electrolytically severable joint being composed of molybdenum (Mo) or an alloy thereof, the alloy having at least one of a Young's modulus greater than 35 Msi, a mechanical strength greater than 400 Ksi, and a magnetic susceptibility less than 300.

2. The vaso-occlusive assembly of claim 1, wherein the alloy has at least two of a Young's modulus greater than 35 Msi, a mechanical strength greater than 400 Ksi, and a magnetic susceptibility less than 300.

3. The vaso-occlusive assembly of claim 1, wherein the alloy has a Young's modulus greater than 35 Msi, a mechanical strength greater than 400 Ksi, and a magnetic susceptibility less than 300.

4. The vaso-occlusive assembly of claim 1, wherein the alloy has a Young's modulus greater than 35 Msi.

5. The vaso-occlusive assembly of claim 1, wherein the alloy has a mechanical strength greater than 400 Ksi.

6. The vaso-occlusive assembly of claim 1, wherein the alloy has a magnetic susceptibility less than 300.

7. The vaso-occlusive assembly of claim 1, wherein the Mo contained in the alloy is greater than 20% in weight.

8. The vaso-occlusive assembly of claim 1, wherein the Mo contained in the alloy is greater than 40% in weight.

9. The vaso-occlusive assembly of claim 1, wherein the electrolytically severable joint is composed of a Mo alloy.

10. The vaso-occlusive assembly of claim 9, wherein the Mo alloy is selected from the group consisting of molybdenum (Mo)-rhenium (Re), molybdenum (Mo)-tungsten (W), molybdenum (Mo)-rhodium (Rh), molybdenum (Mo)-Iridium (Ir), molybdenum (Mo)-platinum (Pt), molybdenum (Mo)-palladium (Pd), molybdenum (Mo)-gold (Au), molybdenum (Mo)-Tantalum (Ta), molybdenum (Mo)-niobium (Nb), molybdenum (Mo)-zirconium (Zr), molybdenum (Mo)-cerium (Ce), hafnium (Hf), and any combination thereof.

11. The vaso-occlusive assembly of claim 10, wherein the Mo alloy is molybdenum (Mo)-rhenium (Re).

12. The vaso-occlusive assembly of claim 11, wherein the Re contained in the Mo alloy is greater than 20% by weight.

13. The vaso-occlusive assembly of claim 11, wherein the Re contained in the Mo alloy is 47.5% by weight.

14. The vaso-occlusive assembly of claim 1, wherein the alloy has an electrical resistivity less than 100μσ·cm.

15. The vaso-occlusive assembly of claim 1, wherein the alloy has an electrochemical potential less than −0.1 V.

16. The vaso-occlusive assembly of claim 1, wherein the vaso-occlusive device has a length greater than 5 cm.

17. The vaso-occlusive assembly of claim 1, wherein the vaso-occlusive device is a vaso-occlusive coil.

18. The vaso-occlusive assembly of claim 1, wherein the delivery catheter has an inner lumen in which the vaso-occlusive device is retrained, the inner lumen having a diameter less than 0.020 inches.

19. A vaso-occlusive treatment system, comprising:
the vaso-occlusive assembly of claim 1; and
the delivery catheter.

20. The vaso-occlusive treatment system of claim 16, further comprising an electrolytic detachment device to which a proximal end of the delivery wire of the vaso-occlusive assembly is configured for being electrically coupled, the electrolytic detachment device configured for delivering electrical current to the electrolytically severable joint of the vaso-occlusive assembly while the vaso-occlusive device is disposed within aneurysmal sac, such that the vaso-occlusive device electrolytically detaches from the distal end of the delivery wire.
